(19) United States
(12) Patent Application Publication
(10) Pub. No.: US 2013/0302840 A1
(43) Pub. Date: Nov. 14, 2013

(54) BLOOD ALCOHOL TEST APPARATUS AND METHOD

(71) Applicant: Earl R. Smith, (US)

(72) Inventors: Earl R. Smith, Volente, TX (US); Barbara A. Yarbrough, Austin, TX (US); Casey P. Reyes, Austin, TX (US)

(21) Appl. No.: 13/737,139

(22) Filed: Jan. 9, 2013

Related U.S. Application Data
(60) Provisional application No. 61/586,347, filed on Jan. 13, 2012.

(51) Int. Cl. C12Q 1/32

(52) U.S. Cl. CPC C12Q 1/32
USPC 435/26

(57) ABSTRACT

The subject matter consists of a small, hand held, portable, battery run device similar to a current diabetic monitor. The device will be carried on a person in a custom protective case that can be attached to a belt, in a pocket or on the waist of a shirt or pants. The monitor test strip will be impregnated with a substance that will be able to detect, with only a small amount of blood (e.g. a drop or a few drops) the BAC of an individual within a relatively short amount of time (e.g. seconds to minutes).

### RELATED APPLICATION

This application claims the priority of U.S. Provisional Application No. 61/586,347 filed on Jan. 13, 2012 and entitled “BLOOD ALCOHOL TEST APPARATUS AND METHOD.”

### FIELD OF THE INVENTION

The invention relates to an apparatus and method for testing the Blood Alcohol Concentration (“BAC”) of an individual.

### BACKGROUND OF THE INVENTION

Blood Alcohol Concentration (“BAC”) is the concentration of alcohol in the blood, expressed as the weight of alcohol in a fixed volume of blood and used as a measure of the degree of intoxication in an individual. The concentration depends on body weight, the quantity and rate of alcohol ingestion, and the rates of alcohol absorption and metabolism. Also referred to throughout as “Blood Alcohol Level.”

Generally, alcohol is metabolized at the rate of 0.015 of BAC every hour. For instance, a person with a BAC of 0.15 (this BAC is almost twice the legal amount when driving in most US locations) will have no measurable alcohol in the bloodstream ten hours after the last drink (0.15 divided by 0.015=10).

Relevant alcohol information and statistics reveal that for employers, alcohol abuse and alcoholism problems account for approximately 67% of total number of substance abuse complaints. In the United States, alcohol abuse or alcoholism is associated with half of the automobile fatalities and almost half of all industrial accidents. Indeed, more and more companies are employing alcohol screening tests and random alcohol tests as part of their workplace drug and alcohol testing program.

Alcohol abuse in the workplace essentially has two main focal points: the first concerns actual drinking on the job and the second concerns working while under the influence of alcohol. Commentators agree that the most efficient way to significantly reduce these types of behavior is the implementation of a random alcohol testing program for all employees.

Consequently, there is a growing demand for more effective alcohol testing, screening, and detection methods in the American workplace; however, employers are faced with few options in considering implementing an alcohol testing program.

There are basically five different types of alcohol tests: (i) urine tests; (ii) blood tests; (iii) saliva tests; (iv) breathalyzer tests; and (v) hair tests.

### Urine Based Alcohol Testing

Urine Alcohol Tests (also called alcohol urine tests) have the following characteristics:

They are the least expensive of the current alcohol testing methods.
The urine sample can be obtained almost anywhere (e.g. at home, in a business restroom, etc.).
They detect alcohol ingestion mainly within the past week, or longer with regular drinking.
Considered relatively reliable
Most likely of drug-testing methods to withstand legal challenge.

However, urine alcohol tests have many drawbacks:

They are considered an intrusive and embarrassing method of alcohol testing because to ensure sample integrity, the person providing the sample is often watched by another person.
They can be affected by abstaining from drinking for a period of time before the test.
It can take up to two hours from alcohol ingestion to appear in the person's urine.
Without supervising the sample collection, the specimen can be adulterated, substituted, or diluted.
They require lab verification for accurate results.
A positive urine test does not necessarily mean the person was under the influence of alcohol at the time of the test. Rather, it detects and measures the use of alcohol within the previous day (longer with special testing methods).
Only indirectly tests BAC (e.g. does not actually test the alcohol in the blood).

### Traditional Blood Alcohol Testing

Traditional blood alcohol testing requires a phlebotomist to draw blood from the person's body and the test reveals the amount of alcohol in the blood at the time the blood sample was taken. Traditional blood alcohol test have the following characteristics:

Blood alcohol testing is considered one of the most accurate methods for testing a person's BAC.
Long history of withstanding legal challenge if blood draw performed properly

However, traditional blood alcohol tests have many drawbacks:

Many common medications can alter the results including non-prescription drugs; therefore, all medications must be disclosed prior to the test.
Very invasive because requires a blood draw from, traditionally, a vein.
Significantly higher risk of infection than other methods.
Generally requires a phlebotomist or other specially trained personnel to obtain the sample.
One of the most expensive ways to test BAC.

### Saliva Alcohol Testing

Saliva alcohol tests detect the presence of alcohol in the saliva and are apparently a relatively good approximation of BAC because the concentration of alcohol in saliva is believed to be similar to the BAC in the blood. Saliva Tests have the following characteristics:

More expensive than urine based tests, but less expensive then blood testing.
Relatively non-intrusive.
They are easy to obtain the sample under supervision.
Minimal risk of tampering with the sample.
Detect use primarily within the past day or so.

However, there are drawbacks to saliva alcohol testing:

They have no nationally accepted cutoff concentrations or standards for detection.
Significantly more susceptible for legal challenge.

### Breathalyzer

Breathalyzer tests are the most common form of breath alcohol testing and are widely used by law enforcement throughout the country. They have the following characteristics:

Relatively non-intrusive.
Relatively easy to obtain the sample after instruction.
Nearly no risk of tampering.
Until recently, not very susceptible to legal challenge.

However, there are significant deficiencies in breathalyzer testing:

Only indirectly tests BAC (e.g. does not actually test the alcohol in the blood).
They not only detect the ethyl alcohol found in alcohol beverages, but also in other substances that have a similar molecular structure.
They can result in false BAC readings caused from cell phones, police radios, electrical interference, moisture, dirt, and tobacco smoke.
They can result in false BAC readings from substances or compounds found in gasoline, paint removers, cleaning fluids, celluloid, and lacquers.
They can lead to false BAC results from blood, vomit, or alcohol present in the person's mouth.
They can result in false BAC readings due to a person's breathing rate caused by vigorous exercise, hyperventilation, or a person holding one's breath.
They can result in false BAC readings if the breathalyzer test is performed improperly or if the unit is not re-calibrated when necessary.

### Hair Alcohol Testing

Interestingly, hair alcohol testing is relatively recent. Before about 2008, hair tests could not detect alcohol and were therefore used only for testing for drugs other than alcohol. As the hair grows, it absorbs special markers called ethyl glucuronide (“EtG”) and fatty acid ethyl esters (“FAEEs”). The EtG and FAEE markers remain in the hair indefinitely as long as a person doesn't shave or cut his or her hair. Because the FAEE and EtG markers are only produced when there is alcohol in the bloodstream, the more markers that exist, the more alcohol the individual has consumed.

Extensive research studies on EtG and FAEE testing have helped researchers establish a reliable base line for FAEEs and EtGs regarding the drinking patterns of different groups of people such as heavy drinkers, social drinkers, and non-drinkers. Since the concentration of blood to body hair fails to elicit reliable results, only scalp hair appears to lead to an accurate alcohol assessment.

Hair Alcohol Tests have the following characteristics:

Relatively non-intrusive method of alcohol testing.
Detects alcohol use over a longer period of time than any other type of alcohol testing protocol (e.g. hair tests for alcohol can provide accurate test results regarding alcohol use going back months or even years).
No adulterants have been found that can beat hair tests for alcohol (e.g. dyes, perms, etc, do not change the results).
Risk of tampering with the sample is minimized because sample collection may be directly and easily observed.
The sample is relatively stable (e.g. it does not deteriorate).
The sample is easy to ship and store (e.g. no refrigeration, not hazardous fluid).

However, hair alcohol tests have many drawbacks:

Many times more expensive than urine testing.
Require a sample of hair approximately the diameter of a pencil and about 1.5 inches long.
Subject to legal challenges because such a new detection method.
Cannot detect whether a person is under the influence at the time of the test.

### BRIEF SUMMARY OF THE INVENTION

The disclosed subject matter provides an apparatus and methods for the accurate detection of BAC directly from the blood of a person without the need for a phlebotomist or other special training.

An aspect of the disclosed subject matter is to provide a portable, hand held, battery operated BAC measurement device.

Another aspect of the disclosed subject matter is to provide disposable test strips for use in the BAC measurement device.

Yet another aspect of the disclosed subject matter is to provide a method and retrofit kit to adapt a diabetic testing device into the BAC measurement device.

Additionally, the subject matter with instant results would revolutionize BAC testing to a more accurate, definitive, less intrusive, less expensive and time effective process.

The subject matter will eliminate the need to transfer a suspect to a facility for a blood draw by a phlebotomist.

The subject matter will streamline arrests.

The subject matter will save costs to taxpayers, city police departments and courts.

The subject matter will assist in keeping the innocent from being arrested and the guilty arrested with valid proof.

The subject matter could improve the percentage of convictions of alcohol related charges by withstanding legal challenge.

The subject device is a portable device that can be carried on a person in a custom, protective sleeve that can be attached to a belt, placed in a pocket or on the waist of pants.

The subject device is less invasive and could reduce the chance of infection and transfer of disease for both the police and the suspect

The subject device would require minimal training to use.

The subject device only requires a minimal amount of blood.

The subject device could be beneficial to probation and parole officers, prison officials, forensic scientists, rehabilitation faculties, entire medical society, private and government employers and the general public to test their own BAC before getting behind the wheel of a car.

These and other aspects of the disclosed subject matter, as well as additional novel features, will be apparent from the description provided herein. The intent of this summary is not to be a comprehensive description of the claimed subject matter, but rather to provide a short overview of some of the subject matter's functionality. Other systems, methods, features and advantages here provided will become apparent to one with skill in the art upon examination of the following detailed description. It is intended that all such additional systems, methods, features and advantages that are included within this description, be within the scope of any claims filed later.

### DETAILED DESCRIPTION OF THE INVENTION

The subject matter consists of a small, hand held, portable, battery run device similar to a current diabetic monitor. The device will be carried on a person in a custom protective case that can be attached to a belt, in a pocket or on the waist of a shirt or pants. The monitor test strip will be impregnated with a substance that will be able to detect, with only a small amount of blood (e.g. a drop or a few drops) the BAC of an individual within a relatively short amount of time (e.g. seconds to minutes).

The finger of the person to be tested would first be disinfected for example by using an alcohol swab. A lancet would be used to pierce the skin of the person's finger. It is important that the lancet be sterile such as a disposable lancet. When the person begins to bleed, a small sample would be applied directly to the test strip. A small adhesive bandage would be applied over the puncture site. The test strip would absorb the blood and the test strip would be inserted into the BAC measurement device. The device will process the blood sample through a series of chemical reactions to determine the BAC. The results will be indicated on the screen of the BAC measurement device.

In one embodiment, the BAC measurement device could have the ability to be programmed to specific legal limits for operating a motor vehicle on roads, rails, water, in the air, over land and underground for different localities. The device will be beneficial for use in the medical field and forensic research.

In yet another embodiment, the BAC measurement device monitor could show the test results in different languages and even provide a printout of the results.

An additional method could include:

Disinfect the finger
Get a test strip and insert into meter
Use the lancet to prick the side of a finger, squeeze gently to expose blood
Make certain meter is on and then apply the drop of blood to the strip
Results will be indicated on the monitor screen in seconds
Disinfect point of lancet entry
Discard used items

Generally, an embodiment of the BAC measurement device will measure the blood alcohol content of an individual citizen in a similar process to a diabetes monitor involving a blood sample, test strips and a monitor. The machine will consists of test strips which contain a capillary in which the blood sample will be collected along with a enzyme (maybe alcohol dehydrogenase enzymes) and proper copper/metal pieces that will transfer the electrical current from the capillary to the monitor once it has been inserted, the monitor device will contain electrical circuits and digital appliances that will read the electrical current from the test strip and digital produce an legible measurement of the individuals BAC. In addition, instead of an enzyme, a light, laser, or chemical reaction may be utilized.

Test Strip: The test strip (similar to that of a diabetic test strip) has a capillary in which the blood sample will be collected. Within the capillary, the enzyme will react with the blood and undergo a chemical reaction. The electrical current of the chemical reaction will be transferred to the monitor by the use the copper metal backbone within the strip. This “backbone” will conduct the electrical current and pass it to the monitor.

Monitor: the monitor will be similar to that of the diabetic testing machine. Consisting of electrical date chips, circuit's digital monitors and a screen. The inserted test strip will pass the current to the monitor. The monitor's digital and electrical circuits will process these currents and deliver the results in a legible manner to the monitor screen. The data chips have been specially customized to read the electrical current and process the data in the proper BAC units.

Lancet: the lancet will puncture the individual's skin and draw the blood to the surface.

Once an individual has been designated to have their blood tested. Remove the monitor, test strips and lancet from proper storage. Turn the monitor on, and insert the test strip. Use the lancet to puncture the skin and draw the blood to the surface. Take the monitor with the inserted test strip and place end of the test strip to the blood. Once the test strip has collected the blood, press the READ button. Within seconds the monitor will read and present the results.

In addition to the above described embodiments, those skilled in the art will appreciate that this disclosure has application in a variety of arts and situations and this disclosure is intended to include the same.

What is claimed is:

1. A method as disclosed above.
